(19) United States
(12) Patent Application Publication
(10) Pub. No.: US 2023/0218073 A1
(43) Pub. Date: Jul. 13, 2023

(54) FINGERPRINT FORENSICS

(71) Applicant: Peter Lee FRYER, Wapadrand (ZA)

(72) Inventor: Peter Lee FRYER, Wapadrand (ZA)

(21) Appl. No.: 18/152,392

(22) Filed: Jan. 10, 2023

(30) Foreign Application Priority Data
Jan. 10, 2022 (ZA) 2022/00415

(51) Int. Cl. A46B 11/00; A46B 5/00; A46B 9/02; A46D 1/00; A46B 15/00; A61B 5/1172

(52) U.S. Cl. CPC A46B 11/0006; A46B 5/0095; A46B 9/02; A46B 11/0072; A46B 15/0036; A46D 1/0207; A61B 5/1172; A46B 2200/20

(57) ABSTRACT

The invention relates a powder brush, which includes a body having mounting means for mounting a powder cartridge thereon and a brush head assembly attachable to an operative front end of the body. The brush head assembly defines a bristle mounting means capable of retaining a plurality of circumferentially arranged bristles therein. Furthermore, the powder brush includes a powder dispensing arrangement, in use, operable selectively to dispense powder from the powder cartridge onto the plurality of bristles. The dispensing arrangement includes an agitator disposed inside the powder cartridge, which cartridge includes a biasing means operable to bias the agitator towards a closed and open position. The bristles comprise of features, in particular, ostrich feathers of which a stem portion is secured to the brush head assembly.

### CROSS-REFERENCE TO RELATED APPLICATIONS

This application claims benefit of South African Provisional Application No. 2022/00415 filed Jan. 10, 2022, which is incorporated herein by reference in its entirety.

### FIELD OF THE INVENTION

This invention relates to fingerprint forensics. In particular, the invention relates to a powder brush.

### BACKGROUND OF THE INVENTION

Conventionally a latent fingerprint and/or palmprint is left on a surface after being touched by a human. The latent fingerprints are made visible by applying a special fine forensic powder to the fingerprint. This has to be done without disturbing or destroying the fingerprint. Application of such powder is often by means of a forensic brush which usually comprises of a handle portion to which a brush head is attached, often comprising of bristles, used to brush the surface.

Brush heads often contain very fine Marabou feathers because of their extremely fine texture. However, often the Marabou feathers are not available or in short supply and a viable alternative should be found.

The use of ostrich feathers, as bristles, has been investigated due to the soft texture thereof. However, an ostrich feather has a rigid spine portion and directional feathers, which might lead to uneven distribution of powder on a latent fingerprint.

In use, usually a skilled person will apply a powder to the surface after which the surface is dusted with the brush, alternatively the powder, which is contained in a separate container, is firstly applied to the brush portion, after which the surface is dusted for uncover prints.

The inventor is aware of existing powder dispensing applicator brushes and the various techniques used by the skilled person.

The inventor has identified a need for an improved powder brush.

### SUMMARY OF THE INVENTION

According to a first aspect of the invention there is provided a powder brush, which includes
a body having mounting means for mounting a powder cartridge thereon; and
a brush head assembly attachable to an operative front end of the body, the brush head assembly defining bristle mounting means capable of retaining a plurality of circumferentially arranged bristles therein.

The powder brush may include a powder dispensing arrangement, in use, operable selectively to dispense powder from the powder cartridge onto the plurality of bristles. The powder dispensing arrangement may be operable to dispense measured doses of powder from the powder cartridge.

The bristles may be feathers. More particularly, the bristles may be ostrich feathers.

The powder brush may include a powder cartridge mountable onto the body. The powder cartridge may be interchangeable.

The body may be tubular. The powder cartridge may be tubular and filled with a forensic powder.

The brush head assembly may include a powder dispensing aperture disposed on an operative front end and may include a powder cartridge mounting portion on an operative rear end thereof. The powder cartridge mounting portion may be threaded. The threaded powder cartridge mounting portion may be matched to a threaded portion on the operative front end of the body, for connecting them together.

The brush head assembly may include a duct extending between the powder cartridge mounting portion and the powder dispensing aperture, for dispensing powder from the powder cartridge onto the plurality of bristles, in use.

In one embodiment, the bristle mounting means may include a flange portion, in which is defined a plurality of slits radially arranged around the powder dispensing aperture.

In another embodiment the bristle mounting means may include a frusto-conical retainer, which includes radially arranged longitudinal slits on an outer surface thereof.

The plurality of radially arranged slits may be recessed from the outer circumference for locating the feathers in a secure manner.

In use, a stem portion of the feathers may be inserted through the slits, thereby securing the feathers to the brush head assembly in a friction fit manner.

The powder dispensing device may include a dispensing arrangement operable selectively to dispense powder from the powder cartridge. The dispensing arrangement may be operable to dispense measured doses of powder from the powder cartridge.

The dispensing arrangement may include an agitator disposed inside the powder cartridge.

The agitator may include a closure operable to cooperate with a front end of the cartridge, selectively to close the front end of the cartridge. In one embodiment the closure may be in the form of a disk at a front end of the agitator operable to close onto a shoulder at an inside of the cartridge front end. The cartridge may include biassing means operable to bias the agitator towards a closed position onto the shoulder.

The disk may include a rotational ratchet mechanism, operable to rotate the agitator when operated.

The disk may include at least one dispensing aperture and the shoulder may include a matched dispensing aperture, which when both apertures are radially in register opens the inside of the cartridge onto the duct in the brush head assembly and, when both apertures are not radially in register, closes the inside of the cartridge onto the duct in the brush head assembly.

The agitator may include a longitudinal planar portion operable to rotate in unison with the rotational ratchet.

The biasing means may include a compression spring and an actuating mechanism. The compression spring may urge the agitator from a rear portion of the cartridge forward towards a closed position onto the shoulder. The disk and shoulder may include two opposed cams operable to rotate the agitator when cooperating with the shoulder. In use, the compression spring retains the agitator in a closed position and the actuating mechanism allows for the rotational movement of the agitator relative to the shoulder.

The actuating mechanism may be in the form of a twist cap, a retractable pen mechanism, or the like.

The powder cartridge may be factory filled with the powder and include a breakable seal, operable to break upon insertion of the powder cartridge within the body.

According to another aspect of the invention, there is provided a powder brush, which includes
a body having mounting means for mounting a powder cartridge thereon; and
a brush head assembly attachable to an operative front end of the body, the brush head assembly defining bristle mounting means capable of retaining a plurality of circumferentially arranged bristles therein; and
a powder dispensing device, operable to dispose powder from the cartridge onto the plurality of bristles.

The powder dispensing device may include an agitator removably inserted into an operative rear end of the cartridge.

The agitator may include an attachment means on an operative rear end thereof, allowing the elongated member to be secured to an operative rear end of the cartridge.

The attachment means may be threaded and matched to a complementary thread on an operative rear end of the cartridge, allowing for screw on attachment and screw off detachment. Advantageously, attachment and detachment of the agitator provides an agitating effect on forensic powder in the cartridge.

The powder brush may include a light source attachable to an outer surface of the brush head assembly, which in use allows for illumination at an operative front end of the plurality of bristles.

The light source may include an electronic circuitry for controlling the lights source and switch for activation thereof.

The light source may comprise of light emitting diodes, or the like.

The invention is now described, by way of non-limiting example, with reference to the accompanying figures:

### FIGURE(S)

In the figure(s):

FIG. 1 shows a three-dimensional view of a powder brush in an assembled formation; and

FIG. 2 shows an exploded view of the powder brush as shown in FIG. 1.

In the figures, like reference numerals denote like parts of the invention unless otherwise indicated.

### EMBODIMENT OF THE INVENTION

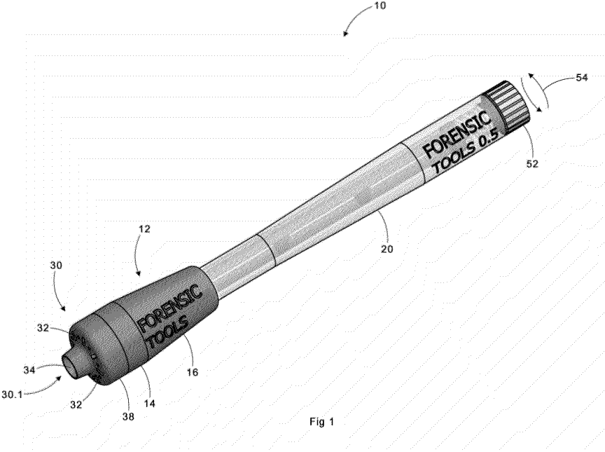

In FIG. 1 reference number (10) refers to powder brush which includes a body (12) having mounting means (not shown) for mounting a powder cartridge (20) to an operative rear end (12.1) of the body (12), a powder cartridge (20) and a brush head assembly (30) attachable to an operative front end (12.2) of the body (12), whereby the brush head assembly (30) defines a bristle mounting means (32) capable of retaining a plurality of circumferentially arranged bristles (not shown) therein.

In this example, the powder cartridge (20) is factory filled with powder and includes a breakable seal (not shown), operable to break upon insertion of an operative front end (20.1) of the powder cartridge (20) within the body (12). Furthermore, the bristles comprise of ostrich feathers.

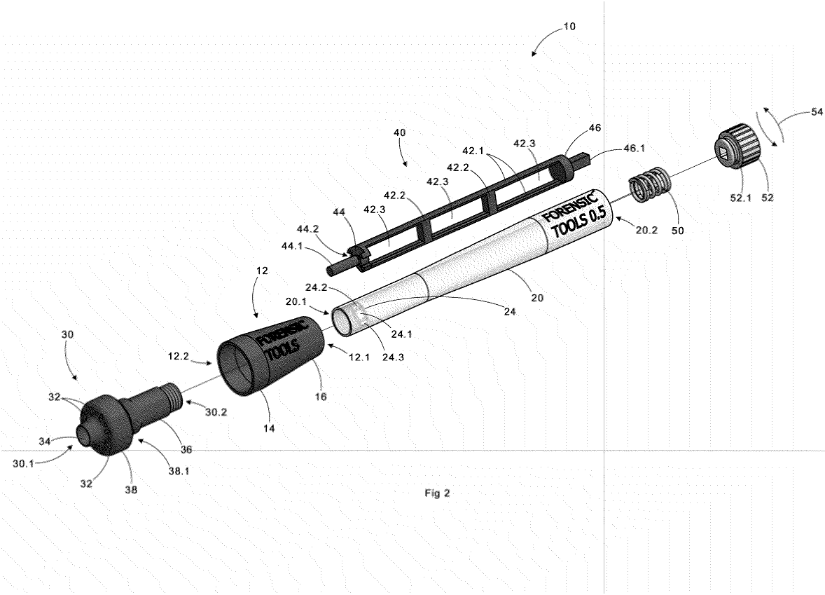

Furthermore, the powder brush (10) includes a powder dispensing arrangement (40), as shown in FIG. 2, which in use is operable selectively to dispense measured doses of powder from the powder cartridge (20) onto the plurality of bristles.

The body (12) comprises of a tubular shape of which the operative front end (12.2) includes a cylindrical portion (14), whereafter the body (12) includes a tapered portion (16) towards the operative rear end (12.1) thereof, defining mounting means.

The brush head assembly (30) includes a powder dispensing aperture (34) disposed on an operative front end (30.1) and a powder cartridge mounting portion (36) on an operative rear end (30.2) thereof. The power dispensing aperture (34) is connected to the powder cartridge mounting portion (36) with a duct, extending therebetween. In use, the duct (34) allows for powder dispensed from the powder cartridge (20) to be move through the duct (34) and onto the bristles.

The bristle mounting means includes a flange portion (38), positioned around the duct (34), in which is defined a plurality of slits (32) radially arranged around the duct (34).

The powder cartridge mounting portion (36) includes a first threaded portion matched to a second threaded portion on an inside surface at an operative front end (20.1) of the powder cartridge (20), allowing for a screw-in connection between the brush head assembly (30) and the powder cartridge (20). During assembly, the first threaded portion is inserted from the operative front end (12.2) of the body (12) and the second threaded portion is inserted from the operative rear end (12.1) of the body (12), whereafter the two threaded portions are connected. It should be appreciated that, during the connection of the two threaded portions, the flange portion (38) will be compression fitted within the body (12) at the tapered portion (16), resulting the body (12) to be secured between the brush head assembly (30) and the powder cartridge (20).

The plurality of radially arranged slits (32) are recessed from the outer circumference of the flange portion (38) for locating the feathers on a secure manner.

In use, a stem portion (not shown) of the feathers is inserted through the slits (32), thereby securing the feathers to the brush head assembly (30) in a friction fit manner. Upon insertion of the stem portions through the slits (32), a lower portion of each stem would be exposed past a rear portion (38.1) of the flange portion (38), however, the cylindrical portion (14) of the body (12) would cover and clamp the stems, once the two threaded portions have been connected. Furthermore, the bristles would extend past the dispensing aperture (34), allowing for dispensed powder to be dispensed onto the bristles.

The powder dispensing arrangement (40) includes an agitator (42) disposed inside the powder cartridge (20).

The agitator (42) includes an elongated member, operable to rotate within the cartridge (20), which comprises of two opposing rods (42.1) connected by two transversely arranged rods (42.2) which divides the elongated member into three equal segments (42.3), thereby operable to engage with the powder within the powder cartridge (20). Furthermore, the elongated member includes two disks (44, 46) on each opposing end thereof, of which the disk (44) is used as a closure.

The cartridge (20) includes a shoulder (24) at an inside of the operative front end (20.1) of the cartridge (20), in use, operable to cooperate with the disk (44), thereby capable of selectively opening and closing the front end (20.1) of the cartridge (20).

The disk (44) includes a longitudinal axle (44.1) and two opposing dispensing apertures (44.2). Furthermore, the disk (46), includes a second longitudinal axle (46.1).

The shoulder (24) includes a guiding aperture (24.1) and a matched dispensing aperture arrangement (24.2, 24.3).

In use, the first longitudinal axle (44.1) is inserted through the guiding aperture (24.1), allowing for the dispensing apertures (44.2 and 24.2, 24.3) to be able to rotate radially relative to each other. Therefore, when the apertures (44.2 and 24.2, 24.3) are radially in register with each other, it would result in an open position and accordingly opens the inside of the cartridge (20) onto the duct (34) in the brush head assembly (30) and, when the apertures (44.2 and 24.2, 24.3) are not radially in register, resulting in a closed position, it closes the inside of the cartridge (20) onto the duct (34) in the brush head assembly (30).

Th cartridge (20) includes a biasing means operable to bias the agitator (42) from the open position towards the closed position.

The biasing means includes a compression spring (50) and an actuating mechanism (52).

The actuating means (52) comprises of a twist cap attachable to a rear portion (20.2) of the cartridge (20), by means of a complementary shaped threaded formation (52.1) on the twist cap (52) and on an inner surface on the rear portion (20.2) of the cartridge (20), allowing for a rotational movement (54) of the twist cap (52) relative to the cartridge (20). Furthermore, the twist cap (52) includes a steering aperture, shaped and dimensioned to receive the second longitudinal axle (46.1) therein, whereby the twist cap (52) is operable to rotate the agitator (42) within the cartridge (20).

In use, the compression spring (50) is placed over the second longitudinal axle (46.1), whereafter the twist cap (52) is attached to the cartridge (20).

Furthermore, the disk (44) and the shoulder (24) include two opposed cams operable to rotate the disk (44) when cooperating with the shoulder (42), resulting in a rotational rachet mechanism.

In use, the compression spring (50) urges the agitator (42) from a rear portion (20.2) of the cartridge (20) forward towards the shoulder (24), thereby retaining the dispensing apertures (44.2 and 24.2, 24.3) in either the open or closed position, whereby the rotational movement (54) of the twist cap (52) will alter the position of the dispensing apertures (44.2 and 24.2, 24.3) from either the open to close position or form the closed to open position, allowing for the dispensing of measured doses of powder from the powder cartridge (20) onto the plurality of bristles.

The inventor believes that the invention provides a novel all-in-one powder dispensing applicator brush device, aimed at simplifying the day-to-day use for a person wanting to capture a latent fingerprint.

1. A powder brush, which includes
a body having mounting means for mounting a powder cartridge thereon; and
a brush head assembly attachable to an operative front end of the body, the brush head assembly defining bristle mounting means for retaining a plurality of circumferentially arranged bristles therein.

2. A powder brush as claimed in claim 1, which includes a powder dispensing arrangement, in use, operable selectively to dispense powder from the powder cartridge onto the plurality of bristles.

3. A powder brush as claimed in claim 2, in which the powder dispensing arrangement is operable to dispense measured doses of powder from the powder cartridge.

4. A powder brush as claimed in claim 1, in which the bristles are ostrich feathers.

5. A powder brush as claimed in claim 1, which includes an interchangeable, tubular powder cartridge mountable onto the body, the powder cartridge being filled with a forensic powder.

6. A powder brush as claimed in claim 1, in which the brush head assembly includes a powder dispensing aperture disposed on an operative front end thereof and a powder cartridge mounting portion on an operative rear end thereof, the brush head assembly including a duct extending between the powder cartridge mounting portion and the powder dispensing aperture, for dispensing powder from the powder cartridge onto the plurality of bristles, in use.

7. A powder brush as claimed in claim 1, which includes a dispensing arrangement operable selectively to dispense measured doses of powder from the powder cartridge.

8. A powder brush as claimed in claim 7, in which the dispensing arrangement includes an agitator disposed inside the powder cartridge.

9. A powder brush as claimed in claim 8, in which the agitator includes a closure in the form of a disk at a front end of the agitator operable to close onto a shoulder at an inside of the cartridge front end.

10. A powder brush as claimed in claim 9, in which the cartridge includes biassing means operable to bias the agitator towards a closed position onto the shoulder, the biassing means including a compression spring and an actuating mechanism, the compression spring, in use, urges the agitator from a rear portion of the cartridge forward towards a closed position onto the shoulder.

11. A powder brush as claimed in claim 10, in which the disk includes a rotational ratchet mechanism, operable to rotate the agitator when operated and at least one dispensing aperture, and the shoulder includes a matched dispensing aperture, which when both apertures are radially in register opens the inside of the cartridge onto the duct in the brush head assembly and, when both apertures are not radially in register, closes the inside of the cartridge onto the duct in the brush head assembly.

12. A powder brush as claimed in claim 11, in which the agitator includes a longitudinal planar portion operable to rotate in unison with the rotational ratchet.

13. A powder brush as claimed in claim 12, in which the disk and shoulder include two opposed cams operable to rotate the agitator when cooperating with the shoulder.

14. A powder brush as claimed in claim 5, in which the powder cartridge is factory filled with the powder and includes a breakable seal, operable to break upon insertion of the powder cartridge into the body.

15. A powder brush, which includes
a body having mounting means for mounting a powder cartridge thereon; and
a brush head assembly attachable to an operative front end of the body, the brush head assembly defining bristle mounting means capable of retaining a plurality of circumferentially arranged bristles therein; and
a powder dispensing device, operable to dispose powder from the cartridge onto the plurality of bristles.

16. A powder brush as claimed in claim 15, in which the powder dispensing device includes an agitator removably inserted into an operative rear end of the cartridge.

17. A powder brush as claimed in claim 16, in which the agitator includes an attachment means on an operative rear end thereof, allowing the elongated member to be secured to an operative rear end of the cartridge.

18. A powder brush as claimed in claim 17, in which the attachment means is threaded and matched to a complementary thread on an operative rear end of the cartridge, allowing for screw on attachment and screw off detachment.

19. A powder brush as claimed in claim 18, which includes a light source attachable to an outer surface of the brush head assembly, which in use allows for illumination at an operative front end of the plurality of bristles.

20. A powder brush as claimed in claim 19, in which the light source comprises of light emitting diodes.
